(19) United States
(12) Patent Application Publication
(10) Pub. No.: US 2008/0058654 A1
(43) Pub. Date: Mar. 6, 2008

(54) Manometer with indication label

(76) Inventor: Hun-Yuan Ke, Taichung City (TW)

Correspondence Address: HUN-YUAN KE, 235 Chung - Ho, Box 8-24, Taipei, omitted

(21) Appl. No.: 11/516,254

(22) Filed: Sep. 6, 2006

(51) Int. Cl. A61B 5/02

(52) U.S. Cl. 600/503

(57) ABSTRACT

A manometer with an indication label comprises a manometer body; a tying stripe; a tying stripe; and an indication label for indicating a position for measuring blood pressures or blood pulse; the indication label being installed at a predetermined position of the manometer body. A length of the indication label is 1.5 centimeter or ⅝ inches. The indication label is indicated with a distance of 1.5 centimeter ⅝ inches from a wrist of a user. The indication label is installed on the tying stripe. The indication label is made of soft cloth or plastics.

### FIELD OF THE INVENTION

The present invention relates to manometers, and particularly to a manometer with an indication label, wherein the indication label serves to indicate an optimum measuring position of the manometer body for increasing the precision in measurement.

### BACKGROUND OF THE INVENTION

People often measure blood pressures or pulses by a manometer for recording the values of the blood pressures and pulses for monitoring the conditions of bodies and providing these values to doctors as a reference in treatment. Thus the doctors can adjust the amount of the patient's drugs.

Thus, many people prepare manometers for monitoring blood pressure in a very long term. However, current manometers do not indicate the positions of users for measuring the blood pressures. Thus the positions for measuring the blood pressure are shifted everyday, even the blood pressure cannot be measured.

### SUMMARY OF THE INVENTION

Accordingly, the primary object of the present invention is to provide a manometer with an indication label, wherein the indication label serves to indicate an optimum measuring position of the manometer body for increasing the precision in measurement.

To achieve above objects, the present invention provides a manometer with an indication label which comprises a manometer body; a tying stripe; a tying stripe; and an indication label for indicating a position for measuring blood pressures or blood pulse; the indication label being installed at a predetermined position of the manometer body. A length of the indication label is 1.5 centimeter or ⅝ inches. The indication label is indicated with a distance of 1.5 centimeter ⅝ inches from a wrist of a user. The indication label is installed on the tying stripe. The indication label is made of soft cloth or plastics.

The various objects and advantages of the present invention will be more readily understood from the following detailed description when read in conjunction with the appended drawing.

### BRIEF DESCRIPTION OF THE DRAWINGS

FIG. 1 is a cross sectional view of the manometer with an indication label of the present invention.

FIG. 2 is a perspective view of the manometer with an indication label of the present invention.

FIG. 3 shows the application of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

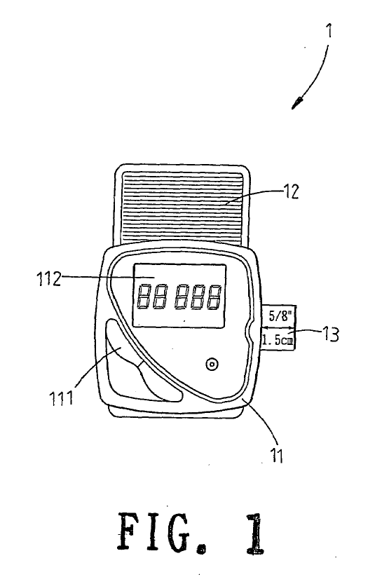

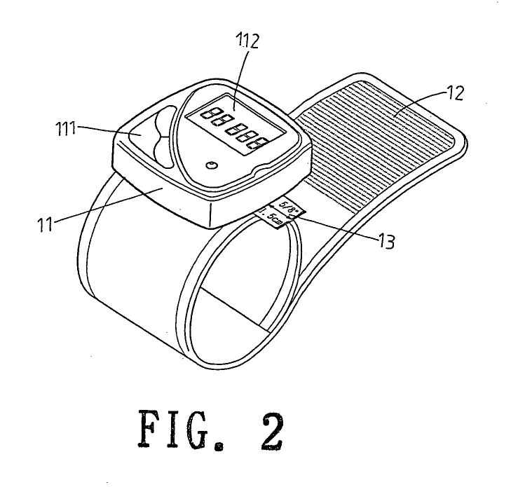

Referring to FIGS. 1 and 2, the present invention has the following elements.

A manometer body 1 has an electronic unit 11, a tying stripe 12 and an indication label 13.

The electronic unit 11 serves to measure the blood pressure and pulses of the blood. The electronic unit 11 has an operation unit 111 and a display 112. The operation unit 111 serves to determine the measuring items about blood pressure and pulses. The measured result is displayed upon the display 112. The electronic unit 11 is fixed to a tying stripe 12.

The tying stripe 12 can be installed to a measure portion of the user for measuring the blood pressure and pulses (the way for measuring the blood pressure is known in the prior art and thus the details will not be further described herein).

The indication label 13 is made of soft cloth or plastics. The indication label 13 is installed on the tying stripe 12 at a position which can be seen conveniently by users, for example, aside the electronic unit 11. The indication label 13 is indicated with 1.5 cm or ⅝ inches for indicating a length of the indication label 13. The indication label 13 serves to indicate an optimum measuring position of the manometer body 1 for increasing the precision in measurement.

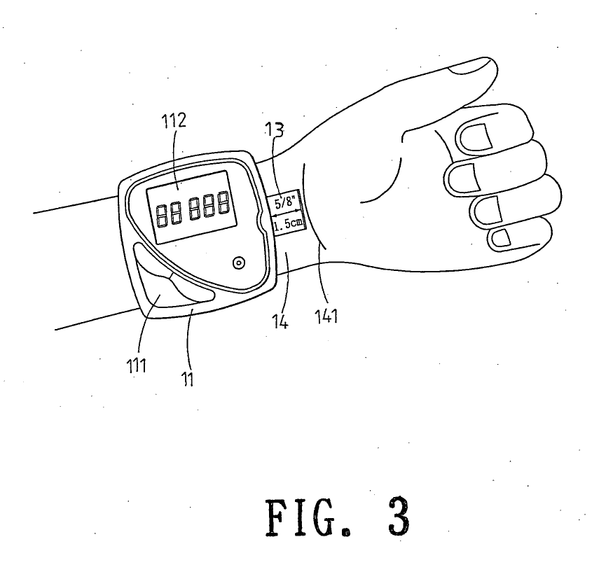

In use of the present invention, referring to FIG. 3, the tying stripe 12 encloses the wrist 14 of a user. Then the one side of the indication label 13 not connecting to the tying stripe 12 is aligned to the joint 141 of the wrist 14 which is an optimum position for measuring the blood pressure. Then the user selects a measuring item by pressing the operation unit 111. When the electronic unit 11 completes the measurement, the measured value will display on the display 112.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A manometer with an indication label comprising:
a manometer body;
a display;
a tying stripe; and
an indication label for indicating a position for measuring blood pressures or blood pulse; the indication label being installed at a predetermined position of the manometer body.

2. The manometer with an indication label as claimed in claim 1, wherein a length of the indication label is 1.5 centimeter.

3. The manometer with an indication label as claimed in claim 1 wherein the indication label is indicated with a distance of 1.5 centimeter from a wrist of a user.

4. The manometer with an indication label as claimed in claim 1, wherein a length of the indication label is ⅝ inches.

5. The manometer with an indication label as claimed in claim 1, wherein the indication label is indicated with a distance of ⅝ inches from a wrist of a user.

6. The manometer with an indication label as claimed in claim 1, wherein the indication label is installed on the tying stripe.

7. The manometer with an indication label as claimed in claim 1, wherein the indication label is made of soft cloth.

8. The manometer with an indication label as claimed in claim 1, wherein the indication label is made of plastics.
